(19) United States
(12) Patent Application Publication
(10) Pub. No.: US 2024/0008772 A1
(43) Pub. Date: Jan. 11, 2024

(54) SYSTEMS AND METHODS FOR OXYGEN LEVEL OR HYDRATION LEVEL SENSING AT VARYING BODY POSITIONS

(71) Applicant: Know Labs, Inc., Seattle, WA (US)

(72) Inventor: Phillip BOSUA, Seattle, WA (US)

(21) Appl. No.: 18/148,491

(22) Filed: Dec. 30, 2022

Related U.S. Application Data
(63) Continuation-in-part of application No. 17/858,437, filed on Jul. 6, 2022.

(51) Int. Cl. A61B 5/145; A61B 5/0507; A61B 5/00

(52) U.S. Cl. CPC A61B 5/14546; A61B 5/0507; A61B 5/4875; A61B 5/14542; A61B 5/0015; A61B 2562/04; A61B 2562/0228

(57) ABSTRACT

One or more analytes are detected at each of at least two different locations on the body by generating a transmit signal in a radio or microwave frequency, transmitting the signal into each of the at least two locations, and measuring a response at each of the at least two locations. One sensor can be used for all of the detections, moving the sensor from one location to another, or a different sensor can be used at each location. Biological parameters including at least one of an oxygen level or a hydration level are determined based on the detection of the one or more analytes at the locations. Differentials between the biological parameters detected at each of the at least two locations can be used to determine conditions in the subject.

### FIELD

This disclosure is directed to sensors and detection methods for biological parameters including oxygen levels and/or hydration levels of a subject at multiple different positions on the subject.

### BACKGROUND

Subjects using non-invasive sensors can experience local variations in analyte levels depending on, for example, the tissue measured, the alignment of the sensor, or the like. Non-invasive sensors can require alignment tools or structures, or measurements using such sensors may include alignment and positioning steps, reducing convenience or comfort of such sensors.

### SUMMARY

This disclosure is directed to sensors and detection methods for biological parameters including oxygen levels and/or hydration levels of a subject at multiple different positions on the subject.

Measurements in variable positions can allow for the detection in local differences in analytes in the subject or differences in biological parameters determined based on such analytes, for example oxygen levels or hydration levels. This can be indicative of subject behavior and/or conditions, such as establishing metabolic activity, detecting aberrant local analyte conditions, determining circulatory or other such issues, and the like. The detection of local conditions can allow, for example, detection of growth of tumor cells based on consumption or generation of particular analytes, measuring or characterizing organ function such as liver or kidney function, and other such detections of conditions associated with differential in levels of that analyte through the body. Measurement in variable positions can also improve convenience to the subject, for example reducing or eliminating the need for consistent positioning while still determining conditions based on detection of analytes in the subject.

In an embodiment, a method of detecting levels of a biological parameter in a subject includes detecting a first level of the biological parameter at a first location on a body of the subject and detecting a second level of the biological parameter at a second location on the body of the subject. The second location is different from the first location. Detecting the first level of the biological parameter includes generating a first transmit signal in a radio or microwave frequency range of the electromagnetic spectrum, transmitting the first transmit signal into the subject at the first location, detecting a first response resulting from transmitting the first transmit signal into the subject at the first location, and processing the first response to determine the first level of the biological parameter. Detecting the second level of the biological parameter includes generating a second transmit signal in a radio or microwave frequency range of the electromagnetic spectrum, transmitting the second transmit signal into the subject at the second location, detecting a second response resulting from transmitting the second transmit signal into the subject at the second location, and processing the second response to determine the second level of the biological parameter. The biological parameter includes at least one of an oxygen level or a hydration level.

In an embodiment, the method further includes determining a condition of the subject based on a differential between the first level of the biological parameter and the second level of the biological parameter. In an embodiment, detecting the first level of the biological parameter is performed using a first sensor and detecting the second level of the biological parameter is performed using a second sensor. In an embodiment, detecting the first level of the biological parameter and detecting the second level of the biological parameter overlap in time. In an embodiment, detecting the first level of the biological parameter includes positioning a sensor at the first location and wherein detecting the second level of the biological parameter includes positioning said sensor at the second location. In an embodiment, the biological parameter includes the oxygen level. In an embodiment, the oxygen level is determined based on the hydration level. In an embodiment, the biological parameter includes the hydration level. In an embodiment, the method further includes detecting a third level of the biological parameter at a third location on the body of the subject, different from the first location and the second location.

In an embodiment, a system for detecting levels of a biological parameter in a subject includes a first sensor. The first sensor includes an antenna array having at least one transmit antenna and at least one receive antenna. The at least one transmit antenna is positioned and arranged to transmit a transmit signal into a target, and the at least one receive antenna is positioned and arranged to detect a response resulting from transmission of the transmit signal by the at least one transmit antenna into the target. The first sensor further includes a transmit circuit that is electrically connectable to the at least one transmit antenna. The transmit circuit is configured to generate a transmit signal to be transmitted by the at least one transmit antenna. The transmit signal is in a radio or microwave frequency range of the electromagnetic spectrum. The first sensor further includes a receive circuit that is electrically connectable to the at least one receive antenna. The receive circuit is configured to receive a response detected by the at least one receive antenna. The system further includes a controller. The controller is configured to process the response detected by the at least one receive antenna at a first location to determine a first level of the biological parameter and receive results of detection of a second level of the biological parameter taken at a second location on the body of the subject. The second location is different from the first location. The controller is further configured to compare the first level to the second level. The biological parameter includes at least one of an oxygen level and a hydration level.

In an embodiment, the system further includes a second sensor. The second sensor includes a second sensor antenna array having at least one second sensor transmit antenna and at least one second sensor receive antenna. The at least one second sensor transmit antenna is positioned and arranged to transmit a second sensor transmit signal into a second target, and the at least one second sensor receive antenna is positioned and arranged to detect a response resulting from transmission of the second sensor transmit signal by the at least one second sensor transmit antenna into the second target. The second target is at the second location on the body of the subject. The second sensor further includes a second sensor transmit circuit that is electrically connectable to the at least one second sensor transmit antenna. The second sensor transmit circuit is configured to generate a second sensor transmit signal to be transmitted by the at least one second sensor transmit antenna. The second sensor transmit signal is in a radio or microwave frequency range of the electromagnetic spectrum. The second sensor also includes a second sensor receive circuit that is electrically connectable to the at least one second sensor receive antenna. The second sensor receive circuit configured to receive the response detected by the at least one second sensor receive antenna. The controller is configured to receive the results of detection of the second level of the biological parameter from the second sensor.

In an embodiment, the controller is configured to receive the results of detection of the second level of the biological parameter from the first sensor. In an embodiment, the controller is configured to determine a condition based on a differential between the first level of the biological parameter and the second level of the biological parameter. In an embodiment, the controller is included in a device including the first sensor. In an embodiment, the controller is included in a remote device, separate from the first sensor. In an embodiment, the controller is included in a cloud server. In an embodiment, the results of detection of the second level of the biological parameter received by the controller include a second amount of each of the one or more analytes detected at the second location, and the controller is further configured to process the second amount to determine the second oxygen level. In an embodiment, the biological parameter includes the oxygen level. In an embodiment, the controller is configured to determine the oxygen level based on the hydration level. In an embodiment, the biological parameter includes the hydration level.

### DRAWINGS

FIG. 1 shows a sensor at a measurement location according to an embodiment.

FIG. 2 shows an analyte detection system according to an embodiment.

FIG. 3 shows a flowchart of a method for detecting one or more analytes according to an embodiment.

FIG. 4 shows a flowchart of a method for determining a condition based on detection of one or more analytes at a plurality of body locations according to an embodiment.

FIG. 5 shows an antenna array of a sensor according to an embodiment.

FIG. 6 shows a flowchart of a method for determining an oxygen level in a subject.

FIG. 7 shows a flowchart of a method for determining a hydration level in a subject.

### DETAILED DESCRIPTION

This disclosure is directed to sensors and detection methods for biological parameters including oxygen levels and/or hydration levels of a subject at multiple different positions on the subject.

The transmit antenna and the receive antenna can be located near the target and operated as further described herein to assist in detecting at least one analyte in the target. The transmit antenna transmits a signal, which includes a frequency in the radio or microwave frequency range, toward and into the target. The signal can be formed by separate signal portions, each having a discrete frequency, that are transmitted separately at separate times at each frequency. In another embodiment, the signal may be part of a complex signal that includes a plurality of frequencies. The complex signal can be generated by blending or multiplexing multiple signals together followed by transmitting the complex signal whereby the plurality of frequencies are transmitted at the same time. One possible technique for generating the complex signal includes, but is not limited to, using an inverse Fourier transformation technique. The receive antenna detects a response resulting from transmission of the signal by the transmit antenna into the target containing the at least one analyte of interest.

The transmit antenna and the receive antenna are decoupled (which may also be referred to as detuned or the like) from one another. Decoupling refers to intentionally fabricating the configuration and/or arrangement of the transmit antenna and the receive antenna to minimize direct communication between the transmit antenna and the receive antenna, preferably absent shielding. Shielding between the transmit antenna and the receive antenna can be utilized. However, the transmit antenna and the receive antenna are decoupled even without the presence of shielding.

The signal(s) detected by the receive antenna can be analyzed to detect the analyte based on the intensity of the received signal(s) and reductions in intensity at one or more frequencies where the analyte absorbs the transmitted signal. Examples of detecting an analyte using a non-invasive spectroscopy sensor operating in the radio or microwave frequency range of the electromagnetic spectrum are described in WO 2019/217461, U.S. Pat. Nos. 11,063,373, 11,058,331, and 11,033,208 the entire contents of which are incorporated herein by reference. The signal(s) detected by the receive antenna can be complex signals including a plurality of signal components, each signal component being at a different frequency. In an embodiment, the detected complex signals can be decomposed into the signal components at each of the different frequencies, for example through a Fourier transformation. In an embodiment, the complex signal detected by the receive antenna can be analyzed as a whole (i.e. without demultiplexing the complex signal) to detect the analyte as long as the detected signal provides enough information to make the analyte detection. In addition, the signal(s) detected by the receive antenna can be separate signal portions, each having a discrete frequency.

In one embodiment, the analyte sensor described herein can be used to detect the presence of at least one analyte in a target. In another embodiment, the analyte sensor described herein can detect an amount or a concentration of the at least one analyte in the target. The target can be any target containing at least one analyte of interest that one may wish to detect. The target can be human or non-human, animal or non-animal, biological or non-biological. For example, the target can include, but is not limited to, human tissue, animal tissue, plant tissue, an inanimate object, soil, a fluid, genetic material, or a microbe. Non-limiting examples of targets include, but are not limited to, one or more of blood, interstitial fluid, cerebral spinal fluid, lymph fluid or urine, human tissue, animal tissue, plant tissue, an inanimate object, soil, genetic material, or a microbe.

The analyte(s) can be any analyte that one may wish to detect. The analyte can be human or non-human, animal or non-animal, biological or non-biological. For example, the analyte(s) can include, but is not limited to, one or more of glucose, blood alcohol, oxygen or an indicator thereof, white blood cells, or luteinizing hormone. The analyte(s) can include, but is not limited to, a chemical, a combination of chemicals, a virus, bacteria, or the like. The analyte can be a chemical included in another medium, with non-limiting examples of such media including a fluid containing the at least one analyte, for example blood, interstitial fluid, cerebral spinal fluid, lymph fluid or urine, human tissue, animal tissue, plant tissue, an inanimate object, soil, genetic material, or a microbe. In an embodiment, the analyte may be simultaneously detected from both blood and interstitial fluid. The analyte(s) may also be a non-human, non-biological particle such as a mineral or a contaminant.

The analyte(s) can include, for example, naturally occurring substances, artificial substances, metabolites, and/or reaction products. As non-limiting examples, the at least one analyte can include, but is not limited to, insulin, acarboxyprothrombin; acylcarnitine; adenine phosphoribosyl transferase; adenosine deaminase; albumin; alpha-fetoprotein; amino acid profiles (arginine (Krebs cycle), histidine/urocanic acid, homocysteine, phenylalanine/tyrosine, tryptophan); andrenostenedione; antipyrine; arabinitol enantiomers; arginase; benzoylecgonine (cocaine); biotinidase; biopterin; c-reactive protein; carnitine; pro-BNP; BNP; troponin; carnosinase; CD4; ceruloplasmin; chenodeoxycholic acid; chloroquine; cholesterol; cholinesterase; conjugated 1-β hydroxy-cholic acid; cortisol; creatine kinase; creatine kinase MM isoenzyme; cyclosporin A; d-penicillamine; de-ethylchloroquine; dehydroepiandrosterone sulfate; DNA (acetylator polymorphism, alcohol dehydrogenase, alpha 1-antitrypsin, cystic fibrosis, Duchenne/Becker muscular dystrophy, analyte-6-phosphate dehydrogenase, hemoglobin and variants thereof including hemoglobin A, hemoglobin S, hemoglobin C, hemoglobin D, hemoglobin E, hemoglobin F, D-Punjab, and beta-thalassemia, particular conformations or conjugations of hemoglobin such as oxyhemoglobin, deoxyhemoglobin, carboxyhemoglobin, and the like; hepatitis B virus; HCMV, HIV-1, HTLV-1, Leber hereditary optic neuropathy, MCAD, RNA, PKU, Plasmodium vivax, sexual differentiation, 21-deoxycortisol); desbutylhalofantrine; dihydropteridine reductase; diptheria/tetanus antitoxin; erythrocyte arginase; erythrocyte protoporphyrin; esterase D; fatty acids/acylglycines; free β-human chorionic gonadotropin; free erythrocyte porphyrin; free thyroxine (FT4); free tri-iodothyronine (FT3); fumarylacetoacetase; galactose/gal-1-phosphate; galactose-1-phosphate uridyltransferase; gentamicin; analyte-6-phosphate dehydrogenase; glutathione; glutathione perioxidase; glycocholic acid; glycosylated hemoglobin; halofantrine; hemoglobin variants; hexosaminidase A; human erythrocyte carbonic anhydrase I; 17-alpha-hydroxyprogesterone; hypoxanthine phosphoribosyl transferase; immunoreactive trypsin; lactate; lead; lipoproteins ((a), B/A-1, β); lysozyme; mefloquine; netilmicin; phenobarbitone; phenytoin; phytanic/pristanic acid; progesterone; prolactin; prolidase; purine nucleoside phosphorylase; quinine; reverse tri-iodothyronine (rT3); selenium; serum pancreatic lipase; sissomicin; somatomedin C; specific antibodies (adenovirus, anti-nuclear antibody, anti-zeta antibody, arbovirus, Aujeszky's disease virus, dengue virus, Dracunculus medinensis, Echinococcus granulosus, Entamoeba histolytica, enterovirus, Giardia duodenalisa, Helicobacter pylori, hepatitis B virus, herpes virus, HIV-1, IgE (atopic disease), influenza virus, Leishmania donovani, leptospira, measles/mumps/rubella, Mycobacterium leprae, Mycoplasma pneumoniae, Myoglobin, Onchocerca volvulus, parainfluenza virus, Plasmodium falciparum, polio virus, Pseudomonas aeruginosa, respiratory syncytial virus, rickettsia (scrub typhus), Schistosoma mansoni, Toxoplasma gondii, Trepenoma pallidium, Trypanosoma cruzi/rangeli, vesicular stomatis virus, Wuchereria bancrofti, yellow fever virus); specific antigens (hepatitis B virus, HIV-1); succinylacetone; sulfadoxine; theophylline; thyrotropin (TSH); thyroxine (T4); thyroxine-binding globulin; trace elements; transferrin; UDP-galactose-4-epimerase; urea; uroporphyrinogen I synthase; vitamin A; white blood cells; zinc protoporphyrin; prostaglandins such as PGF2α and PGE2; hormones such as estrogen, progesterone, and/or follicle stimulating hormone (FSH).

The analyte(s) can also include one or more chemicals introduced into the target. The analyte(s) can include a marker such as a contrast agent, a radioisotope, or other chemical agent. The analyte(s) can include a fluorocarbon-based synthetic blood. The analyte(s) can include a drug or pharmaceutical composition, with non-limiting examples including ethanol or other alcohols; ketones; cannabis (marijuana, tetrahydrocannabinol, hashish); inhalants (nitrous oxide, amyl nitrite, butyl nitrite, chlorohydrocarbons, hydrocarbons); cocaine (crack cocaine); stimulants (amphetamines, methamphetamines, Ritalin, Cylert, Preludin, Didrex, PreState, Voranil, Sandrex, Plegine); depressants (barbiturates, methaqualone, tranquilizers such as Valium, Librium, Miltown, Serax, Equanil, Tranxene); hallucinogens (phencyclidine, lysergic acid, mescaline, peyote, psilocybin); narcotics (heroin, codeine, morphine, opium, meperidine, Percocet, Percodan, Tussionex, Fentanyl, Darvon, Talwin, Lomotil); designer drugs (analogs of fentanyl, meperidine, amphetamines, methamphetamines, and phencyclidine, for example, Ecstasy); anabolic steroids; and nicotine. The analyte(s) can include other drugs or pharmaceutical compositions. The analyte(s) can include neurochemicals or other chemicals generated within the body, such as, for example, ascorbic acid, uric acid, dopamine, noradrenaline, 3-methoxytyramine (3MT), 3,4-Dihydroxyphenylacetic acid (DOPAC), Homovanillic acid (HVA), 5-Hydroxytryptamine (5HT), and 5-Hydroxyindoleacetic acid (FHIAA).

In an embodiment, the analyte(s) are one or more analytes that can be used to determine an oxygen level in a subject. The analytes can be, for example, elemental oxygen, oxyhemoglobin, deoxyhemoglobin, or any other suitable analyte indicative of or a proxy for the oxygen level in the subject. The oxygen level can be an overall level of oxygen or analyte(s) indicative of or a proxy for oxygen by itself, or can be a ratio such as a ratio of oxyhemoglobin to deoxyhemoglobin.

In an embodiment, the analyte(s) can include one or more indicators for determination of hydration of a subject. The analyte(s) can include, for example, hemoglobin, red blood cells as a whole, one or more hormones, sodium, one or more solutes from which osmolarity can be determined, or the like. The amount of the analyte(s) can be used to determine one or more indicia of hydration, such as concentrations of one or more analytes, hematocrit, osmolarity, or any other suitable measurement of a hydration level of the subject. The osmolarity can be an osmolarity of one or more of plasma, interstitial fluid, saliva, urine, or the like. In an embodiment, a sensor can be positioned such that the results of detection are indicative of the presence or amount of analytes in the bladder of the subject, such that urine parameters related to hydration such as urine osmolarity can be determined. In an embodiment, the sensor can be positioned such that results of detection are indicative of the presence or amount of analytes in saliva. A hydration level can be determined based on the one or more indicators, for example by comparing osmolarity or hematocrit to reference values. The reference values can be reference values specific to the subject, general reference values, reference values for a group that the subject belongs to, or the like. In an embodiment, the sensor can detect the one or more analytes in the subject non-invasively. In an embodiment, the sensor can detect the one or more analytes in a sample obtained from the subject, such as a blood, urine, or saliva sample. The sample can have a predetermined mass or volume.

In an embodiment, the sensor described herein can be incorporated into a wearable device such as a ring, a watch, or any other suitable wearable device that is worn on the user's body. The wearable device may be configured to be worn by the user over a longer period of time, for example a watch, a ring, or the like. Alternatively, the wearable device may be configured to be temporarily worn, for example only during one or more analyte readings after which the wearable device is removed. In an embodiment, the sensor described herein can be configured as a non-wearable device. For example, the sensor can be configured as a device that a user holds or presses against a body part during an analyte reading, or a body part is pressed against the sensor, during an analyte reading.

The device including the sensor, whether wearable or non-wearable, can also be configured to be capable of detecting one or more physiological parameters such as user heart rate, user blood pressure, user body temperature, user calorie consumption, user glucose level, one or more hormone levels, bioelectric impedance, or the like. One or more of the physiological parameters can be detected directly using the sensor and/or determined based on detection of one or more analytes by the sensor. In an embodiment, one or more of the physiological parameters can be detected or determined using one or more additional physiological sensors included in the device in addition to the sensor described herein. The one or more additional physiological sensors can be any suitable physiological sensor for the particular physiological parameter to be sensed. In an embodiment, one or more of the physiological parameters can be determined based on a presence or amount of one or more analytes detected by the sensor and one or more additional measurements made by one or more additional physiological sensors included in the device. The device can also include one or more additional functionalities including, but not limited to, a camera; an accelerometer; a pedometer; a fitness/activity tracker; an altimeter; a barometer; a compass; a global positioning system; a sleep monitor; a fall sensor; a microphone; a speaker; and others.

In an embodiment, the one or more analyte(s) can be detected in some or all of a plurality of tissues, bodily fluids, and the like that are subjected to a transmit signal, in turn resulting in a response signal. For example, a transmit signal into a subject at a location where the transmit signal passes through, for example, skin, bone, muscle, interstitial fluid, blood vessels, and blood can result in a response signal indicative of the presence and/or amount of analytes present in some or all of the tissues and/or fluids that the transmit signal enters into or passes through. In embodiments, the response signal can be indicative of the presence and/or amount of some or all of a plurality of organs, such as the liver, the pancreas, the kidneys, the gallbladder, and/or any other such organ. In an embodiment, the response signal can be parsed based on characteristics thereof to estimate or determine the presence and/or amount of the analyte(s) in particular tissues and/or bodily fluids, for example the presence and/or amount of the analyte specifically present in blood, interstitial fluid, a particular tissue or organ, or the like.

Non-invasive detection of the one or more analyte(s), an oxygen level, and/or a hydration level can be performed at multiple different locations on the body. Different locations can be locations that are at various distinct positions on the body of the subject. In an embodiment, different locations can be defined by being on at least different sections of the body, with such sections of the body being, for example, the lower left leg, upper left leg, lower right leg, upper right leg, lower left arm, upper left arm, groin, abdomen, chest, neck, and/or the head.

In an embodiment, at least one of the different locations can be selected such that a particular organ or tissue is subjected to the transmit signal and thus contributes to the response signal. Non-limiting examples of such organs include the liver, the pancreas, the heart, the brain, and the lungs. Non-limiting examples of such particular tissues can include specific muscles, blood flowing through specific regions of the body or specific blood vessels, interstitial fluid at locations of interest, for example at locations of edema, or the like. In this embodiment, another of the different locations can be a location selected to determine a baseline for the one or more analyte(s) also being detected at the particular organ or tissue. Examples of baselines for the analytes can be determined in blood, interstitial fluid, muscle or other tissue, or the like, at a location separate from the particular organ or tissue selected for at least one of the different locations. In embodiments, the detection of the one or more analytes at the particular organ or tissue can be compared to the detection of the one or more analytes at the baseline, for example to determine a differential between an amount of analyte(s) at the particular organ or tissue and the amount of analyte(s) of the baseline. Such differentials can be used to determine conditions, for example tumor growth based on factors such as glucose consumption, metabolite generation, or any other suitable such factor, excessive local concentrations of particular analytes at organs or tissues of interest, digestive or microbiome issues, properties of analytes such as ability to cross blood-brain, placental, intestinal or other barriers, or any other condition or property producing local differentials in one or more analytes.

In an embodiment, the different locations can be selected for measurement of the one or more analyte(s) based on availability, comfort for the subject, convenience for the subject, or the like at a time of the measurement. For example, it may be convenient for a subject to detect the one or more analytes using a sensor positioned at a first position, such as the wrist of the subject during some activities, while other activities may make such sensor positioning inconvenient, uncomfortable, or otherwise unfeasible. Accordingly, detection of the analyte(s) can be performed at a second location other than the first location, for example at an ankle of the subject, on the opposite wrist of the subject, or the like when that is more feasible for the subject. In embodiments, the detection of the one or more analytes can be adjusted according to the location where the detection is performed, for example to account for variance in levels of the analyte based on differences in expected analyte levels at the different locations such as order or amount of blood flow to the location where the measurement is taken, or the like. In an embodiment, accounting for variance in levels of the analyte based on the measurement location can be based on historical data for the subject. In an embodiment, accounting for variance in levels of the analyte based on the measurement location can be based on a database including historical data from a plurality of subjects. In an embodiment, accounting for variance in levels of the analyte based on the measurement location can be based on a model for determining expected differences in detected analyte levels by location, for example models of blood flow and/or analyte consumption or generation at particular locations in the body. Non-limiting examples of variance that can be accounted for by adjustment of detected analyte levels include differences in oxygen levels between left and right arms, differences in glucose between measurements on the torso and measurements on extremities, and the like.

In an embodiment, detection of the one or more analytes at each of the different locations can be performed using one sensor, with the detection at each of the different locations being at separate times. In such an embodiment, the one sensor can be positioned at a first location, and subsequently moved from the first location to a second location, and so on to perform the detection at each of the different locations. In an embodiment, detection of the one or more analytes at each of the different locations can be performed using a plurality of sensors. In such an embodiment, the detection of the one or more analytes at each of the different locations can each be separate in time, or detection of the one or more analytes at two or more of the different locations can be performed at overlapping times.

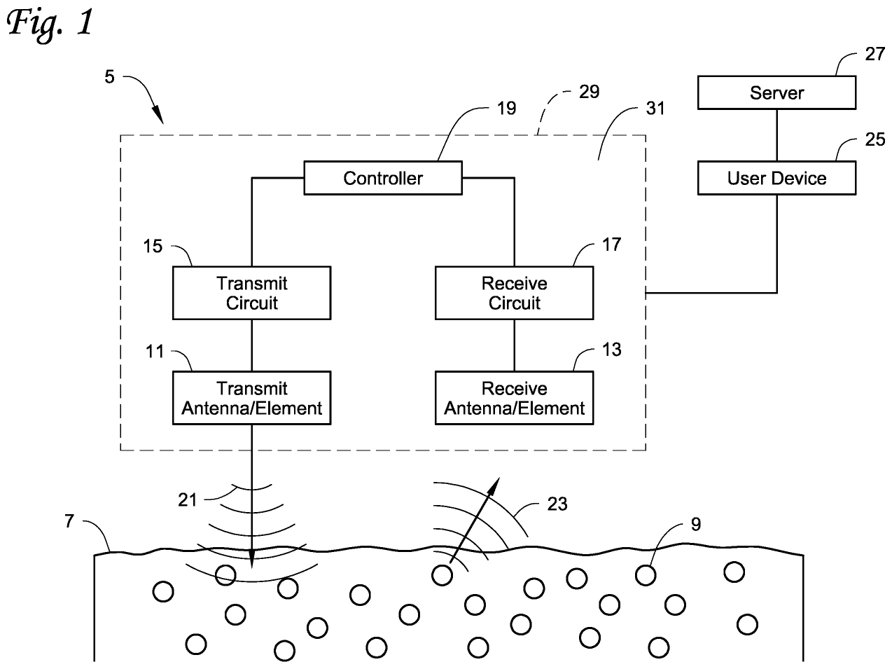

FIG. 1 shows a sensor at a measurement location according to an embodiment. An embodiment of a non-invasive analyte sensor system with a non-invasive analyte sensor 5 is illustrated. The sensor 5 is depicted relative to a target 7 that contains one or more analyte(s) of interest 9. The one or more analyte(s) of interest can include one or more analytes indicative of an oxygen level of a subject, such as, for example, elemental oxygen, oxyhemoglobin, deoxyhemoglobin, and/or any other suitable indicator or proxy for an oxygen level in the subject. In this example, the sensor 5 is depicted as including an antenna array that includes a transmit antenna/element 11 (hereinafter “transmit antenna 11”) and a receive antenna/element 13 (hereinafter “receive antenna 13”). The sensor 5 further includes a transmit circuit 15, a receive circuit 17, and a controller 19. As discussed further below, the sensor 5 can also include a power supply, such as a battery (not shown in FIG. 1).

The target 7 can be one of the plurality of different locations at which the analyte(s) of interest 9 are to be detected. For example, the target 7 can be for example, the lower left leg, upper left leg, lower right leg, upper right leg, lower left arm, upper left arm, groin, abdomen, chest, neck, and/or the head. In an embodiment, the target 7 can be selected such that a particular organ or tissue is subjected to the transmit signal and thus contributes to the response signal. Non-limiting examples of such organs include the liver, the pancreas, the heart, and the lungs. Non-limiting examples of such particular tissues can include specific muscles, blood flowing through specific regions of the body or specific blood vessels, interstitial fluid at locations of interest, for example at locations of edema, or the like. In an embodiment, the target 7 can be selected to provide a baseline against which to compare the measurement of the particular organ or tissue, for example at a location including one or more of blood, interstitial fluid, muscle or other tissue, and/or the like, at a location separate from the particular organ or tissue. In an embodiment, the target 7 can be on a part of the body selected for the comfort or convenience of the subject.

The transmit antenna 11 is positioned, arranged and configured to transmit a signal 21 that is the radio frequency (RF) or microwave range of the electromagnetic spectrum into the target 7. The transmit antenna 11 can be an electrode or any other suitable transmitter of electromagnetic signals in the radio frequency (RF) or microwave range. The transmit antenna 11 can have any arrangement and orientation relative to the target 7 that is sufficient to allow the analyte sensing to take place. In one non-limiting embodiment, the transmit antenna 11 can be arranged to face in a direction that is substantially toward the target 7.

The signal 21 transmitted by the transmit antenna 11 is generated by the transmit circuit 15 which is electrically connectable to the transmit antenna 11. The transmit circuit 15 can have any configuration that is suitable to generate a transmit signal to be transmitted by the transmit antenna 11. Transmit circuits for generating transmit signals in the RF or microwave frequency range are well known in the art. In one embodiment, the transmit circuit 15 can include, for example, a connection to a power source, a frequency generator, and optionally filters, amplifiers or any other suitable elements for a circuit generating an RF or microwave frequency electromagnetic signal. In an embodiment, the signal generated by the transmit circuit 15 includes a frequency in the range from about 10 kHz to about 100 GHz. In another embodiment, the frequency can be in a range from about 300 MHz to about 6000 MHz. In an embodiment, the transmit circuit 15 can be configured to sweep through a range of frequencies that are within the range of about 10 kHz to about 100 GHz, or in another embodiment a range of about 300 MHz to about 6000 MHz. In an embodiment, the transmit circuit 15 can be configured to produce a complex transmit signal, the complex signal including a plurality of signal components, each of the signal components having a different frequency. The complex signal can be generated by blending or multiplexing multiple signals together followed by transmitting the complex signal whereby the plurality of frequencies are transmitted at the same time.

The receive antenna 13 is positioned, arranged, and configured to detect one or more electromagnetic response signals 23 that result from the transmission of the transmit signal 21 by the transmit antenna 11 into the target 7 and impinging on the analyte(s) 9. The receive antenna 13 can be an electrode or any other suitable receiver of electromagnetic signals in the radio frequency (RF) or microwave range. In an embodiment, the receive antenna 13 is configured to detect electromagnetic signals including a frequency in the range from about 10 kHz to about 100 GHz, or in another embodiment a range from about 300 MHz to about 6000 MHz. The receive antenna 13 can have any arrangement and orientation relative to the target 7 that is sufficient to allow detection of the response signal(s) 23 to allow the analyte sensing to take place. In one non-limiting embodiment, the receive antenna 13 can be arranged to face in a direction that is substantially toward the target 7.

The receive circuit 17 is electrically connectable to the receive antenna 13 and conveys the received response from the receive antenna 13 to the controller 19. The receive circuit 17 can have any configuration that is suitable for interfacing with the receive antenna 13 to convert the electromagnetic energy detected by the receive antenna 13 into one or more signals reflective of the response signal(s) 23. The construction of receive circuits are well known in the art. The receive circuit 17 can be configured to condition the signal(s) prior to providing the signal(s) to the controller 19, for example through amplifying the signal(s), filtering the signal(s), or the like. Accordingly, the receive circuit 17 may include filters, amplifiers, or any other suitable components for conditioning the signal(s) provided to the controller 19. In an embodiment, at least one of the receive circuit 17 or the controller 19 can be configured to decompose or demultiplex a complex signal, detected by the receive antenna 13, including a plurality of signal components each at different frequencies into each of the constituent signal components. In an embodiment, decomposing the complex signal can include applying a Fourier transform to the detected complex signal. However, decomposing or demultiplexing a received complex signal is optional. Instead, in an embodiment, the complex signal detected by the receive antenna can be analyzed as a whole (i.e. without demultiplexing the complex signal) to detect the analyte as long as the detected signal provides enough information to make the analyte detection.

The controller 19 controls the operation of the sensor 5. The controller 19, for example, can direct the transmit circuit 15 to generate a transmit signal to be transmitted by the transmit antenna 11. The controller 19 further receives signals from the receive circuit 17. The controller 19 can optionally process the signals from the receive circuit 17 to detect the analyte(s) 9 in the target 7. In an embodiment, the controller 19 can be configured to determine an oxygen level based on the detection of the analyte(s) 9. The oxygen level can be any suitable indicator of oxygen in the subject in which the analyte(s) 9 are detected, for example an SpO₂ value, a concentration of oxygen, an amount of oxygen, a concentration of oxyhemoglobin, an amount of oxyhemoglobin, a ratio of oxyhemoglobin to deoxyhemoglobin and/or carboxyhemoglobin, or the like. In one embodiment, the controller 19 may optionally be in communication with at least one external device 25 such as a user device and/or a remote server 27, for example through one or more wireless connections such as Bluetooth, wireless data connections such a 4G, 5G, LTE or the like, or Wi-Fi. If provided, the external device 25 and/or remote server 27 may process (or further process) the signals that the controller 19 receives from the receive circuit 17, for example to detect the analyte(s) 9. If provided, the external device 25 may be used to provide communication between the sensor 5 and the remote server 27, for example using a wired data connection or via a wireless data connection or Wi-Fi of the external device 25 to provide the connection to the remote server 27. In an embodiment, the external device 25 and/or the remote server 27 can determine the oxygen level based on the presence and/or amount of the analyte(s) 9 detected by the sensor 5. In an embodiment, the controller 19 is further configured to determine an action to be taken in response to detection of the analyte or analytes of interest 9. In an embodiment, another controller (not shown) separate from controller 19 can determine the action.

With continued reference to FIG. 1, the sensor 5 may include a sensor housing 29 (shown in dashed lines) that defines an interior space 31. Components of the sensor 5 may be attached to and/or disposed within the housing 29. For example, the transmit antenna 11 and the receive antenna 13 are attached to the housing 29. In some embodiments, the antennas 11, 13 may be entirely or partially within the interior space 31 of the housing 29. In some embodiments, the antennas 11, 13 may be attached to the housing 29 but at least partially or fully located outside the interior space 31. In some embodiments, the transmit circuit 15, the receive circuit 17 and the controller 19 are attached to the housing 29 and disposed entirely within the sensor housing 29.

The receive antenna 13 is decoupled or detuned with respect to the transmit antenna 11 such that electromagnetic coupling between the transmit antenna 11 and the receive antenna 13 is reduced. The decoupling of the transmit antenna 11 and the receive antenna 13 increases the portion of the signal(s) detected by the receive antenna 13 that is the response signal(s) 23 from the target 7, and minimizes direct receipt of the transmitted signal 21 by the receive antenna 13. The decoupling of the transmit antenna 11 and the receive antenna 13 results in transmission from the transmit antenna 11 to the receive antenna 13 having a reduced forward gain (S21) and an increased reflection at output (S 22) compared to antenna systems having coupled transmit and receive antennas.

In an embodiment, coupling between the transmit antenna 11 and the receive antenna 13 is 95% or less. In another embodiment, coupling between the transmit antenna 11 and the receive antenna 13 is 90% or less. In another embodiment, coupling between the transmit antenna 11 and the receive antenna 13 is 85% or less. In another embodiment, coupling between the transmit antenna 11 and the receive antenna 13 is 75% or less.

Any technique for reducing coupling between the transmit antenna 11 and the receive antenna 13 can be used. For example, the decoupling between the transmit antenna 11 and the receive antenna 13 can be achieved by one or more intentionally fabricated configurations and/or arrangements between the transmit antenna 11 and the receive antenna 13 that is sufficient to decouple the transmit antenna 11 and the receive antenna 13 from one another.

For example, in one embodiment described further below, the decoupling of the transmit antenna 11 and the receive antenna 13 can be achieved by intentionally configuring the transmit antenna 11 and the receive antenna 13 to have different geometries from one another. Intentionally different geometries refers to different geometric configurations of the transmit and receive antennas 11, 13 that are intentional. Intentional differences in geometry are distinct from differences in geometry of transmit and receive antennas that may occur by accident or unintentionally, for example due to manufacturing errors or tolerances.

Another technique to achieve decoupling of the transmit antenna 11 and the receive antenna 13 is to provide appropriate spacing between each antenna 11, 13 that is sufficient to decouple the antennas 11, 13 and force a proportion of the electromagnetic lines of force of the transmitted signal 21 into the target 7 thereby minimizing or eliminating as much as possible direct receipt of electromagnetic energy by the receive antenna 13 directly from the transmit antenna 11 without traveling into the target 7. The appropriate spacing between each antenna 11, 13 can be determined based upon factors that include, but are not limited to, the output power of the signal from the transmit antenna 11, the size of the antennas 11, 13, the frequency or frequencies of the transmitted signal, and the presence of any shielding between the antennas. This technique helps to ensure that the response detected by the receive antenna 13 is measuring the analyte(s) 9 and is not just the transmitted signal 21 flowing directly from the transmit antenna 11 to the receive antenna 13. In some embodiments, the appropriate spacing between the antennas 11, 13 can be used together with the intentional difference in geometries of the antennas 11, 13 to achieve decoupling.

In one embodiment, the transmit signal (or each of the transmit signals) can be transmitted over a transmit time that is less than, equal to, or greater than about 300 ms. In another embodiment, the transmit time can be than, equal to, or greater than about 200 ms. In still another embodiment, the transmit time can be less than, equal to, or greater than about 30 ms. The transmit time could also have a magnitude that is measured in seconds, for example 1 second, 5 seconds, 10 seconds, or more. In an embodiment, the same transmit signal can be transmitted multiple times, and then the transmit time can be averaged. In another embodiment, the transmit signal (or each of the transmit signals) can be transmitted with a duty cycle that is less than or equal to about 50%.

Further information on the sensor 5 and its components and variations thereof can be found in U.S. Pat. Nos. 11,063,373, 11,234,619, 11,031,970, 11,223,383, 11,058,317, 11,058,331, 11,193,923, 10,548,503, 11,330,997, 11,033,208, 11,234,618, 11,284,819, and 11,284,820, the entire contents of which are incorporated herein by reference in their entirety.

In an embodiment, the sensor 5 can be incorporated into a wearable device such as a ring, a watch, or any other suitable wearable device. For example, housing 29 can be provided on or in the wearable device. The wearable device including sensor 5 can be capable of detecting physiological parameters such as heart rate, blood pressure, oxygen level, hydration level, body temperature, calorie consumption, glucose level, one or more hormone levels, or the like. One or more of the physiological parameters can be detected using sensor 5 or determined based on detection of one or more analytes by the sensor 5. In an embodiment, one or more of the physiological parameters can be detected or determined using another sensor included in the wearable device in addition to the sensor 5. This other sensor can be any suitable sensor for the particular physiological parameter. In an embodiment, one or more of the physiological parameters can be determined based on a presence or amount of one or more analytes detected by the sensor 5 and one or more additional measurements made by another sensor included in the wearable device.

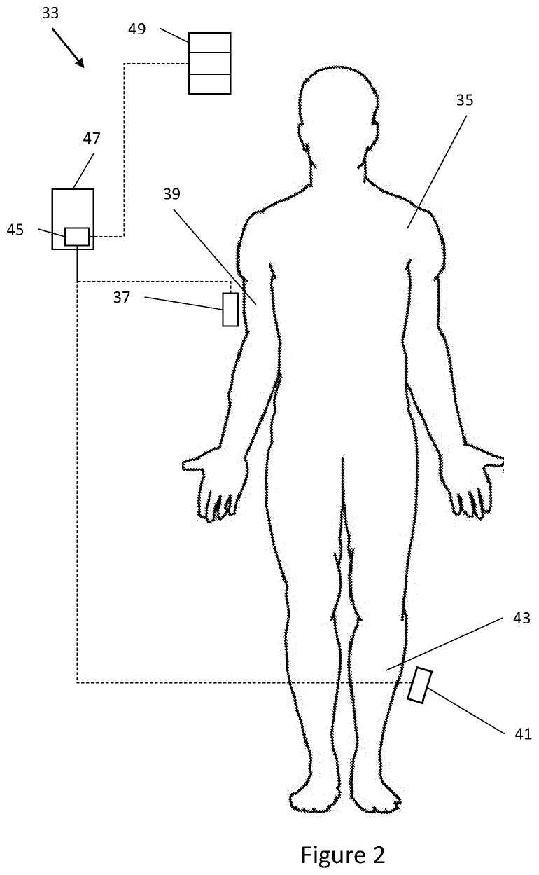

FIG. 2 shows an analyte detection system according to an embodiment. Analyte detection system 33 detects one or more analytes in a subject 35. Analyte detection system 33 includes first sensor 37 positioned to detect one or more analytes at a first location 39. Optionally, a second sensor 41 can be used to detect the one or more analytes at second location 43. In an embodiment, first sensor 37 can be positioned at the second location 43 to perform detection of the one or more analytes. Controller 45 is configured to receive data from first sensor 37 and optionally from second sensor 41. Analyte detection system 33 can include an external device 47 and/or a remote server 49.

Analyte detection system 33 is configured to detect one or more analytes at a plurality different positions on subject 35. The one or more analytes can be any analytes or combination thereof as described above. Subject 35 is any suitable living creature where analytes can be detected at two or more distinct locations. In an embodiment, the subject 35 is a human being. The different positions on subject 35 are distinct locations on the subject 35 that are separate from one another. The distinct locations on the subject 35 can be distinct sections of the body such as, for example, the lower left leg, upper left leg, lower right leg, upper right leg, lower left arm, upper left arm, groin, abdomen, chest, neck, and/or the head. One of these distinct locations on the subject 35 can be each of the first location 39, the second location 43, and/or any further locations where the one or more analytes can be detected in the subject 35.

First sensor 37 is a non-invasive sensor configured to transmit a transmit signal into the subject 35 and obtain a response, so as to detect one or more analytes in the subject. First sensor 37 can be a sensor 5 as described above and shown in FIG. 1. First sensor 37 can be used to detect the one or more analytes at a first location 39 on the subject 35. The first location 39 can be any one of the sections of the body of the subject, such as the lower left leg, upper left leg, lower right leg, upper right leg, lower left arm, upper left arm, groin, abdomen, chest, neck, and/or the head. In an embodiment, the first location 39 is selected based on a convenience or comfort of placing first sensor 37 at the first location 39, for example based on a form factor of the first sensor 37, retention or alignment of the first sensor 37, actions being taken at, near, or using the first location 39 on the subject 35, or any other such suitable factor for placement and/or use of the first sensor 37 at first location 39. In an embodiment, the first location 39 can be selected to correspond to an organ or tissue of interest, such that at least a portion of the response obtained by the first sensor 37 is indicative of the presence or amount of the one or more analytes in the organ or tissue of interest. Non-limiting examples of organs of interest include the liver, the pancreas, the heart, the brain, and the lungs. Non-limiting examples of tissues of interest can include specific muscles, blood flowing through specific regions of the body or specific blood vessels, interstitial fluid at locations of interest, for example at locations of edema, or the like. In an embodiment, the first location 39 can be selected to provide a baseline measurement for comparison to measurement at the second location 43 or further other locations where the one or more analytes can be detected. Examples of locations which can be used as first location 39 to obtain baselines for the analytes include blood, interstitial fluid, muscle or other tissue, combinations thereof, or the like.

In an embodiment, a second sensor 41 can be included in analyte detection system. The second sensor 41 is a non-invasive sensor configured to transmit a transmit signal into the subject 35 and obtain a response, so as to detect one or more analytes in the subject. Second sensor 41 can be a sensor 5 as described above and shown in FIG. 1. In an embodiment, second sensor is identical to first sensor 37. In an embodiment, first sensor 37 and second sensor 41 each have the same form factor, including, for example, size, shape, and/or features for positioning, alignment, and/or retention. In an embodiment, second sensor 41 has a different form factor from first sensor 37. In an embodiment, second sensor 41 can include different features for positioning, alignment, and/or retention of the second sensor 41 to the subject. In embodiments, further sensors can be included in addition to first sensor 37 and second sensor 41. Where sensors in addition to first sensor 37 are included, the various sensors can be operated simultaneously, during overlapping periods of time, or can be operated sequentially at separate times.

Analyte detection system 33 is further configured to detect the one or more analytes as second location 43 on the subject 35. The second location 43 is different and distinct from the first location 39 on the subject 35. Different and distinct locations can be defined as being on at least different sections of the body, with such sections of the body being, for example, the lower left leg, upper left leg, lower right leg, upper right leg, lower left arm, upper left arm, groin, abdomen, chest, neck, and/or the head. In an embodiment, first sensor 37 can be moved so as to be positioned at the second location 43 to perform detection of the one or more analytes following detection of the one or more analytes at the first location 39. In an embodiment, analyte detection system 33 includes second sensor 41 that is used to detect the one or more analytes at the second location 43. In an embodiment, the second location 43 is selected based on a convenience or comfort of placing first sensor 37 or second sensor 41 at the second location 43, for example based on a form factor of the first sensor 37 or second sensor 41, retention or alignment of the first sensor 37 or second sensor 41, actions being taken at, near, or using the second location 43 on the subject 35, or any other such suitable factor for placement and/or use of the first sensor 37 or second sensor 41 at second location 43. In an embodiment, the second location 43 can be selected to correspond to an organ or tissue of interest, such that at least a portion of the response obtained by the first sensor 37 is indicative of the presence or amount of the one or more analytes in the organ or tissue of interest. Non-limiting examples of organs of interest include the liver, the pancreas, the heart, the brain, and the lungs. Non-limiting examples of tissues of interest can include specific muscles, blood flowing through specific regions of the body or specific blood vessels, interstitial fluid at locations of interest, for example at locations of edema, or the like. In an embodiment, the second location 43 can be selected to provide a baseline measurement for comparison to measurement at the first location 39 or further other locations where the one or more analytes can be detected. Examples of locations which can be used as second location 43 to obtain baselines for the analytes include blood, interstitial fluid, muscle or other tissue, combinations thereof, or the like. In embodiments, detection of the one or more analytes can be performed at additional different locations on the subject 35, such as a third location, a fourth location, and so on. In embodiments, one or both of first sensor 37 or optionally second sensor 39 can be moved to such additional locations on the subject 35 to perform detection of the one or more analytes at those additional locations. In embodiments, further additional sensors such as a third sensor, a fourth sensor, and so on can be used to detect the one or more analytes at the additional different locations on the subject 35.

Controller 45 is configured to receive data regarding the detection of the one or more analytes at first location 39, second location 43, and optionally any additional different locations on the subject 35 from the first sensor 37 and optionally from the second sensor 41 or any other additional sensors. The controller 45 can be configured to determine a presence and/or an amount of the one or more analytes from the data received from the detection at the first location 39 and the second location 43. The controller 45 can further be configured to determine an oxygen level based on the presence and/or amount of the one or more analytes detected at the first location 39 and/or the second location 43. The oxygen level can be any suitable indicator of oxygen in the subject in which the analyte(s) are detected, for example an SpO₂ value, a concentration of oxygen, an amount of oxygen, a concentration of oxyhemoglobin, an amount of oxyhemoglobin, a ratio of oxyhemoglobin to deoxyhemoglobin and/or carboxyhemoglobin, or the like. In an embodiment, the controller 45 is configured to adjust an amount of at least some of the one or more analytes detected at first location 39, second location 43, or optionally any additional different locations on the subject 35. In an embodiment, the controller 45 is configured to adjust the determined oxygen level based on detection of the one or more analytes at the first location 39, second location 43, and/or any optional additional location on the subject 35. The adjustment of the amount of the one or more analytes and/or the oxygen level can be based on location-based differences in amounts of the at least some of the one or more analytes or location-based differences in oxygen level. Non-limiting examples of location-based differences in amounts of the one or more analytes or oxygen level include an order or amount of blood flow to the location where the measurement is taken, or the like. In an embodiment, adjustment of the amount of analytes detected or the oxygen level at a location can be based on the measurement location can be based on historical data for the subject. In an embodiment, adjustment of the amount of analytes detected or the oxygen level at a location can be based on a database including historical data from a plurality of subjects. In an embodiment, accounting for variance in amounts of the analyte or the oxygen level based on the measurement location can be based on a model for determining expected differences in detected analyte amounts or oxygen level by location, for example models of blood flow and/or analyte consumption or generation at particular locations in the body. For example, the adjustment of the amount of the one or more analytes or the oxygen level can be to reduce or remove location-related differences in amounts of the one or more analytes, such that results of the detection of the one or more analytes or the oxygen level can be presented on a similar scale or as comparable values.

In an embodiment, controller 45 is configured to determine a condition of the subject 35 or a property of the one or more analytes based on the detection of the one or more analytes or oxygen levels at each of the first location 39, second location 43, and optionally any additional different locations on subject 35. For example, the controller 45 can be configured to compare the results of detection of the one or more analytes or oxygen level at the first location 39 with the results of detection of the one or more analytes or oxygen level at the second location 43. The comparison, such as a differential between the amount of the one or more analytes at the first location 39 and the amount of the one or more analytes at the second location 43, can be used to determine the condition or property. Non-limiting examples of the condition or property include for example tumor growth based on factors such as glucose consumption, metabolite generation, or any other suitable such factor, excessive local concentrations of particular analytes or oxygen levels at organs or tissues of interest, digestive or microbiome issues, properties of analytes such as ability to cross blood-brain, placental, intestinal or other barriers, or any other condition or property producing local differentials in one or more analytes.

In the embodiment shown in FIG. 2, the controller 45 is included in external device 47, which is separate from first sensor 37 and second sensor 45. External device 47 can be, for example, a base station, a computer such as a laptop or desktop computer, a mobile device such as a smart phone or tablet, or any other such device. In an embodiment, controller 45 is included in first sensor 37. In an embodiment, controller 45 is included in second sensor 41. In an embodiment, the controller 45 is included in a remote server 49, which can be, for example, a cloud server. Any or all of first sensor 37, second sensor 41, external device 47, and/or remote server 49 can communicate with one another by one or more wireless connections such as Bluetooth, wireless data connections such a 4G, 5G, LTE or the like, Wi-Fi, and/or wired connections.

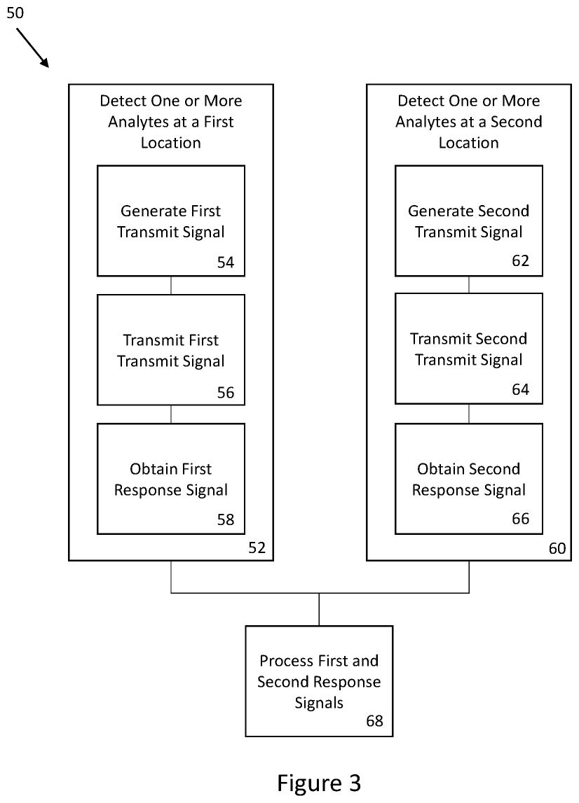

FIG. 3 shows a flowchart of a method for detecting one or more analytes according to an embodiment. Method 50 includes detecting the one or more analytes at a first location on a subject 52. The detection of the one or more analytes at the first location can include generating a first transmit signal 54, transmitting the first transmit signal into the subject at the first location 56, and obtaining a first response signal 58 resulting from the transmitting of the first transmit signal. Method 50 further includes detecting the one or more analytes at a second location on the subject 60. Detecting the one or more analytes at the second location can include generating a second transmit signal 62, transmitting the second transmit signal into the subject at the second location 64, and obtaining a second response signal 66 resulting from the transmitting of the first transmit signal. Method 50 can further include processing at least one of the first response signal and the second response signal to determine a presence and/or amount of the one or more analytes 68.

The one or more analytes are detected at the first location 52. The first location can be any location on a body of the subject, for example a section of the body. Non-limiting examples of sections of the body that can be selected as the first location include the lower left leg, upper left leg, lower right leg, upper right leg, lower left arm, upper left arm, groin, abdomen, chest, neck, or the head. The first location can be selected based on convenience, comfort, or availability of the location on the subject. The first location can be selected to correspond to an organ or tissue of interest, such as the liver, the pancreas, the heart, the brain, the lungs, specific muscles, blood flowing through specific regions of the body or specific blood vessels, interstitial fluid at locations of interest, for example at locations of edema, or the like. The first location can be selected to provide a baseline measurement for comparison to the detection of the one or more analytes at the second location at 60.

Detection of the one or more analytes at the first location can include generating a first transmit signal 54. The first transmit signal can be generated at 54 by a transmit circuit of a sensor, such as transmit circuit 15 of sensor 5 as described above and shown in FIG. 1. The detection of the one or more analytes at the first location at 52 can also include transmitting the first transmit signal into the subject at the first location 56. The first transmit signal can be transmitted into the first location by a transmit antenna of the sensor, such as transmit antenna 11 as described above and shown in FIG. 1. Detection of the one or more analytes at the first location at 52 can further include obtaining a first response signal 58. The first response signal results from the transmitting of the first transmit signal into the subject at the first location. The first response signal can be obtained using a receive antenna of the sensor such as receive antenna 13 of sensor 5 as described above and shown in FIG. 1.

The one or more analytes are detected at the second location 60. The second location can be any distinct location on the subject different from the first location where the one or more analytes are detected at 52. In an embodiment, the detection of the one or more analytes at the second location at 60 can be performed using the same sensor used for detection of the one or more analytes at the first location at 52. In an embodiment, the detection of the one or more analytes at the second location at 60 can be performed using a different sensor from the sensor used for detection of the one or more analytes at the first location at 52. The second location can be selected based on convenience, comfort, or availability of the location on the subject. The second location can be selected to correspond to an organ or tissue of interest, such as the liver, the pancreas, the heart, the brain, the lungs, specific muscles, blood flowing through specific regions of the body or specific blood vessels, interstitial fluid at locations of interest, for example at locations of edema, or the like. The second location can be selected to provide a baseline measurement for comparison to the detection of the one or more analytes at the first location at 52. Detection of the one or more analytes at the second location 60 can include generating a second transmit signal 62. The second transmit signal can be generated at 62 by a transmit circuit of a sensor, such as transmit circuit 15 of sensor 5 as described above and shown in FIG. 1.

The detection of the one or more analytes at the second location at 60 can also include transmitting the second transmit signal into the subject at the second location 64. The second transmit signal can be transmitted into the second location by a transmit antenna of the sensor, such as transmit antenna 11 as described above and shown in FIG. 1. Detection of the one or more analytes at the second location at 60 can further include obtaining a second response signal 66. The second response signal results from the transmitting of the second transmit signal into the subject at the second location. The second response signal can be obtained using a receive antenna of the sensor such as receive antenna 13 of sensor 5 as described above and shown in FIG. 1.

In an embodiment, detection of the one or more analytes at the first location at 52 and detection of the one or more analytes at the second location at 60 are performed at times that overlap with one another. In an embodiment, the detection of the one or more analytes at the first location at 52 and the detection of the one or more analytes at the second location at 60 can be at separate times. In an embodiment, the detection of the one or more analytes at the first location at 52 and the detection of the one or more analytes at the second location at 60 can be performed sequentially.

At least one of the first response signal and the second response signal are processed to determine a presence and/or amount of the one or more analytes 68. In an embodiment, the processing at 68 is performed by a controller such as controller 45 as described above and shown in FIG. 2. The processing at 68 can include processing to convert the received response signal(s) to values indicative of a presence and/or amount of the one or more analytes and/or an oxygen level. The oxygen level can be any suitable indicator of oxygen in the subject in which the one or more analytes are detected, for example an SpO₂ value, a concentration of oxygen, an amount of oxygen, a concentration of oxyhemoglobin, an amount of oxyhemoglobin, a ratio of oxyhemoglobin to deoxyhemoglobin and/or carboxyhemoglobin, or the like. In an embodiment, the processing at 68 can include adjustment of a value indicative of the amount of the one or more analytes and/or an oxygen level, based on the location at which the response signal being processed was obtained. The adjustment of the amount of the one or more analytes or the oxygen level that can be performed at 68 can be based on location-based differences in amounts of the at least some of the one or more analytes or the oxygen level. Non-limiting examples of location-based differences in amounts of the one or more analytes or the oxygen level include an order or amount of blood flow to the location where the measurement is taken, or the like. In an embodiment, adjustment of the amount of analytes detected or the oxygen level at a location can be based on the measurement location can be based on historical data for the subject. In an embodiment, adjustment of the amount of analytes detected or the oxygen level at a location can be based on a database including historical data from a plurality of subjects. In an embodiment, adjusting amounts of the analyte or the oxygen level based on the measurement location can be based on a model for determining expected differences in detected analyte amounts by location, for example models of blood flow and/or analyte consumption or generation at particular locations in the body. For example, the adjustment of the amount of the one or more analytes or the oxygen level can reduce or remove location-related differences in amounts of the one or more analytes, such that results of the detection of the one or more analytes can be presented on a similar scale or as comparable values.

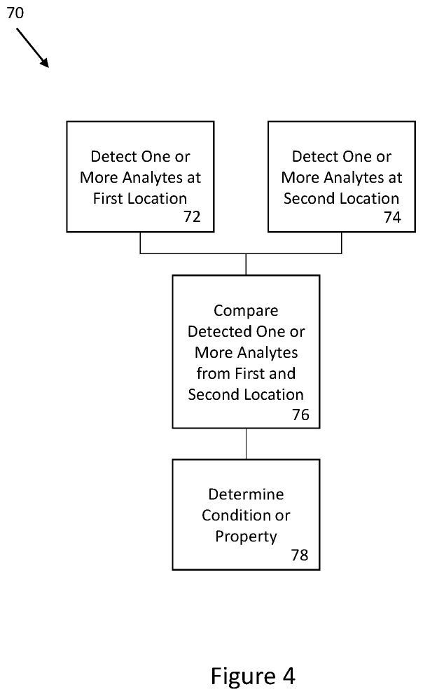

FIG. 4 shows a flowchart of a method for determining a condition based on detection of one or more analytes at a plurality of body locations according to an embodiment. Method 70 includes detecting one or more analytes at a first position on a subject 72, detecting the one or more analytes at a second position on the subject 74, comparing the presence and/or amount of the one or more analytes detected at the first position on the subject with the presence and/or amount of the one or more analytes detected at the second position on the subject 76, and determining a condition or property based on the comparison 78.

The one or more analytes are detected at a first position on a subject at 72. The first position on the subject can be selected to correspond to an organ or tissue of interest, such as the liver, the pancreas, the heart, the brain, the lungs, specific muscles, blood flowing through specific regions of the body or specific blood vessels, interstitial fluid at locations of interest, for example at locations of edema, or the like. The first location can alternatively be selected to provide a baseline measurement for comparison to the detection of the one or more analytes at the second location at 74. The detection of the one or more analytes can be performed according to the detection of the one or more analytes at the first location 52 as shown in FIG. 3 and described above.

The one or more analytes are detected at a second position on the subject 74. The second location can be any distinct location on the subject different from the first location where the one or more analytes are detected at 72. In an embodiment, the detection of the one or more analytes at the second location at 74 can be performed using the same sensor used for detection of the one or more analytes at the first location at 72. In an embodiment, the detection of the one or more analytes at the second location at 74 can be performed using a different sensor from the sensor used for detection of the one or more analytes at the first location at 72. The second position on the subject can be selected to correspond to an organ or tissue of interest, such as the liver, the pancreas, the heart, the brain, the lungs, specific muscles, blood flowing through specific regions of the body or specific blood vessels, interstitial fluid at locations of interest, for example at locations of edema, or the like. The second location can alternatively be selected to provide a baseline measurement for comparison to the detection of the one or more analytes at the first location at 72. The detection of the one or more analytes can be performed according to the detection of the one or more analytes at the second location 60 as shown in FIG. 3 and described above.

In an embodiment, detection of the one or more analytes at the first location at 72 and the detection of the one or more analytes at the second location 74 can be performed at overlapping times. In an embodiment, detection of the one or more analytes at the first location at 72 and the detection of the one or more analytes at the second location 74 can be performed at separate times. In an embodiment, detection of the one or more analytes at the first location at 72 and the detection of the one or more analytes at the second location 74 can be performed sequentially.

The presence and/or amount of the one or more analytes detected at the first position on the subject with the presence and/or amount of the one or more analytes detected at the second position on the subject are compared at 76. In an embodiment, the comparison is to determine if there is a difference in the presence and/or amount of the one or more analytes detected at the first and second locations. In an embodiment, the comparison is to determine whether a greater amount of the one or more analytes was detected at the first location or the second location. In an embodiment, the comparison is to determine an extent of difference in the amount of the one or more analytes detected at the first and second locations. In an embodiment, one or both of the amount of the analytes detected at the first or second location can be adjusted based on a location at which the respective amount was detected. Adjustment of the amount of the one or more analytes can be based on location-based differences in amounts of the at least some of the one or more analytes. Non-limiting examples of location-based differences in amounts of the one or more analytes include an order or amount of blood flow to the location where the measurement is taken, or the like. In an embodiment, adjustment of the amount of analytes detected at a location can be based on the measurement location can be based on historical data for the subject. In an embodiment, adjustment of the amount of analytes detected at a location can be based on a database including historical data from a plurality of subjects. In an embodiment, adjusting the amount of the analyte based on the measurement location can be based on a model for determining expected differences in detected analyte amounts by location, for example models of blood flow and/or analyte consumption or generation at particular locations in the body. For example, the adjustment of the amount of the one or more analytes can reduce or remove location-related differences in amounts of the one or more analytes, such that results of the detection of the one or more analytes can be presented on a similar scale or as comparable values. In embodiments, results of detection of the one or more analytes at additional different locations from the first or second locations can further be included in the comparison performed at 76.

A condition or property is determined based on the comparison at 78. In an embodiment, the comparison is used to determine a condition of the subject. The condition can be based on the particular one or more analytes and the relationship thereof determined by the comparison at 76. The condition can further be based on the particular first and second locations where the one or more analytes are detected at 72 and 74. Non-limiting examples of conditions that can be determined at 78 include tumor growth, impairments to liver function, impairments to kidney function, and other such detections of conditions associated with differential in levels of that analyte through the body. In an embodiment, the condition or property can be determined based on similarity between the detected amounts or trends over time for analytes detected at the first and second locations. For example, consistent levels or consistent trends or patterns in variation in one or more analytes at each of two or more body locations can be indicative of system-wide conditions or characteristics of the subject. For example, where amounts of the analytes vary periodically with similar or the same periodicity to the variance at both the first and second locations, conditions producing body-wide systematic variations, such as heart rates, respiration rates, and the like can be determined based on the levels of the one or more analytes. The particular one or more analytes can be selected based on the condition to be determined. For example, particular metabolites, hormones, signaling compounds, or the like associated with the condition can be included among the one or more analytes that are detected, compared, and used to determine the condition in method 70. In an embodiment, the one or more analytes includes glucose when the condition results in location-specific differences in glucose consumption or release in the body. In an embodiment, the one or more analytes include compounds normally filtered, metabolized, or removed from the body, for example by the liver or kidneys. In an embodiment, instead of a condition of a subject, properties of the one or more analytes can be determined at 78 based on the comparison at 76 of the analytes detected at the first and second locations 72, 74. Examples of such properties include the ability to pass certain barriers in the subject, such as the blood-brain barrier, the placenta, the intestinal walls, or the like. The properties can be determined by suitable location-specific differences, such as a difference between an amount of analyte in the brain compared to the amount of the analyte in other parts of the body being indicative of ability to pass the blood-brain barrier. Other properties of the one or more analytes that can be determined at 78 can include locations where the one or more analytes are processed by the body.

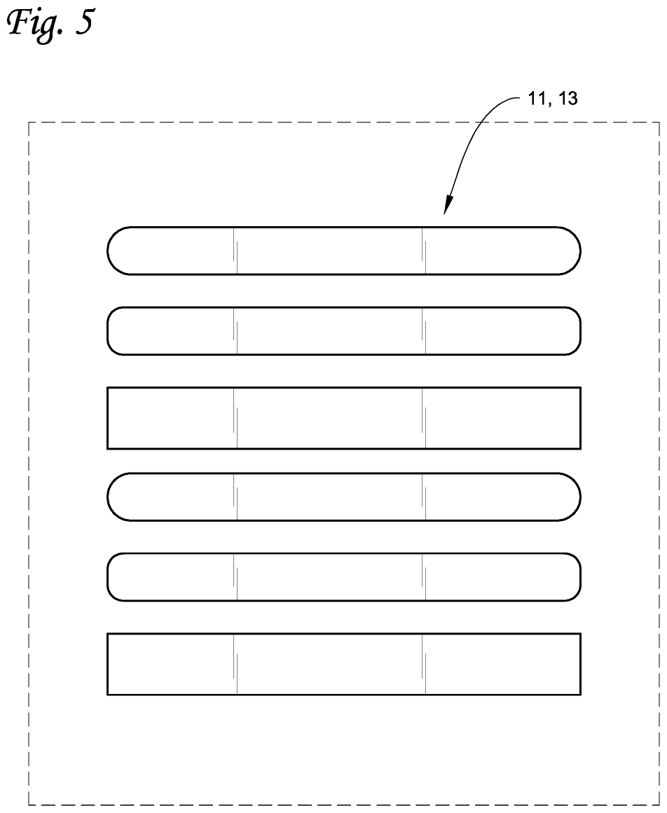

FIG. 5 shows an antenna array of a sensor according to an embodiment. In the array of FIG. 5 illustrates a plan view of an antenna array having six transmit or receive antennas 11, 13. As shown in FIG. 5, the antennas can be substantially linear strips. In this example, the antennas 11, 13 differ in geometry from one another in that the shapes of the ends of the antennas, the lateral lengths and/or the lateral widths of the antennas differ from one another. As shown in the example of FIG. 5, the antennas 11, 13 are arranged side-by-side with one another. The antennas 11, 13, as shown in FIG. 5 have longitudinal axes that are parallel to one another.

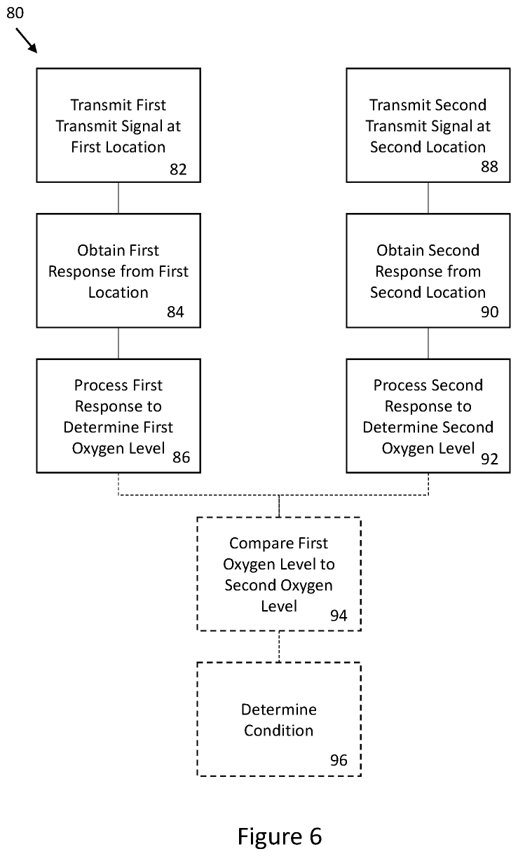

FIG. 6 shows a flowchart of a method for determining an oxygen level at a plurality of locations in a subject. Method 80 includes transmitting a first transmit signal into the subject at a first location at 82, obtaining a first response signal from the first location at 84, and processing the first response signal to determine an oxygen level at the first location at 86. Method 80 further includes transmitting a second transmit signal into the subject at a second location at 88, obtaining a second response signal from the second location at 90, and processing the second response signal to determine an oxygen level at the second location at 92. Method 80 can further include comparing the oxygen level at the first location and the oxygen level at the second location at 94 and determining a condition of the subject based on the comparison at 96.

An oxygen level is determined at a first location by transmitting a first transmit signal into the subject at a first location at 82, obtaining a first response signal from the first location at 84, and processing the first response signal to determine the oxygen level at the first location at 86. The first location can be any location on a body of the subject, for example a section of the body. Non-limiting examples of sections of the body that can be selected as the first location include the lower left leg, upper left leg, lower right leg, upper right leg, lower left arm, upper left arm, groin, abdomen, chest, neck, or the head. The first location can be selected based on convenience, comfort, or availability of the location on the subject. The first location can be selected to correspond to an organ or tissue of interest, such as the liver, the pancreas, the heart, the brain, the lungs, specific muscles, blood flowing through specific regions of the body or specific blood vessels, or the like. The first location can be selected to provide a baseline measurement for comparison to the detection of the oxygen level at the second location. The first transmit signal can be generated by a transmit circuit of a sensor, such as transmit circuit 15 of sensor 5 as described above and shown in FIG. 1 and then transmitted into the subject at the first location 82. The first transmit signal can be transmitted into the first location by a transmit antenna of the sensor, such as transmit antenna 11 as described above and shown in FIG. 1. A first response signal is obtained from the first location on the subject at 84. The first response signal results from the transmitting of the first transmit signal into the subject at the first location 82. The first response signal can be obtained 84 using a receive antenna of the sensor such as receive antenna 13 of sensor 5 as described above and shown in FIG. 1.

The first response signal obtained at 84 is processed to determine a first oxygen level at the first location at 86. In an embodiment, the first response signal obtained at 84 is processed at 86 to determine an amount of oxygen or oxyhemoglobin detected at the first location. In an embodiment, the first response signal obtained at 84 is processed at 86 to determine amounts of one or more analytes used to determine the oxygen level at the first location. The one or more analytes can be, for example, oxygen, carbon dioxide, oxyhemoglobin, deoxyhemoglobin, carboxyhemoglobin, or any other suitable analytes that can be used in a determination of the oxygen level. The amounts of the one or more analytes can in turn be processed to determine the oxygen level, for example by determining a ratio of oxyhemoglobin to deoxyhemoglobin and carboxyhemoglobin, or any other suitable processing to determine the oxygen level. In an embodiment, the processing to determine the oxygen level can be determined and/or adjust based at least in part on the hydration level of the subject. In an embodiment, the hydration level of the subject can be determined based on any one or more suitable analytes during determination of the oxygen level at the first location. In an embodiment, the hydration level can be a hydration level detected at and/or determined for the first location on the subject.

An oxygen level is determined at a second location by transmitting a second transmit signal into the subject at a second location at 88, obtaining a second response signal from the second location at 90, and processing the second response signal to determine a second oxygen level at the second location at 92. The second location can be any distinct location on the subject different from the first location. The second location can be selected based on convenience, comfort, or availability of the location on the subject. The second location can be selected to correspond to an organ or tissue of interest, such as the liver, the pancreas, the heart, the brain, the lungs, specific muscles, blood flowing through specific regions of the body or specific blood vessels, or the like. The second location can be selected to provide a baseline measurement for comparison to the detection of the oxygen level at the first location. The second transmit signal can be generated by a transmit circuit of a sensor, such as transmit circuit 15 of sensor 5 as described above and shown in FIG. 1 and then transmitted into the subject at the second location 88. The second transmit signal can be transmitted into the second location by a transmit antenna of the sensor, such as transmit antenna 11 as described above and shown in FIG. 1. A second response signal is obtained from the second location on the subject at 90. The second response signal results from the transmitting of the second transmit signal into the subject at the second location 88. The second response signal can be obtained 90 using a receive antenna of the sensor such as receive antenna 13 of sensor 5 as described above and shown in FIG. 1.

The second response signal obtained at 90 is processed to determine a second oxygen level at the second location at 92. In an embodiment, the second response signal obtained at 90 is processed at 92 to determine an amount of oxygen or oxyhemoglobin detected at the second location. In an embodiment, the second response signal obtained at 90 is processed at 92 to determine amounts of one or more analytes used to determine the oxygen level at the second location. The one or more analytes can be the same analytes as used to determine the oxygen level at the first location. The one or more analytes can be, for example, oxygen, carbon dioxide, oxyhemoglobin, deoxyhemoglobin, carboxyhemoglobin, or any other suitable analytes that can be used in a determination of the oxygen level. The amounts of the one or more analytes can in turn be processed to determine the oxygen level, for example by determining a ratio of oxyhemoglobin to deoxyhemoglobin and carboxyhemoglobin, or any other suitable processing to determine the oxygen level. In an embodiment, the processing to determine the oxygen level can be determined and/or adjust based at least in part on the hydration level of the subject. In an embodiment, the hydration level of the subject can be determined based on any one or more suitable analytes during determination of the oxygen level at the second location. In an embodiment, the hydration level can be a hydration level detected at and/or determined for the second location on the subject.

In an embodiment, the determination of the oxygen level at the second location can be performed using the same sensor used for determination of the oxygen level at the first location. In an embodiment, the determination of the oxygen level at the second location can be performed using a different sensor from the sensor used for determination of the oxygen level at the first location.

In an embodiment, determination of the oxygen level at the first location and the determination of the oxygen level at the second location can be performed at overlapping times. In an embodiment, determination of the oxygen level at the first location and the determination of the oxygen level at the second location can be performed at separate times. In an embodiment, determination of the oxygen level at the first location and the determination of the oxygen level at the second location can be performed sequentially.

The oxygen level at the first location and the oxygen level at the second location can be compared at 94. In an embodiment, the comparison is to determine if there is a difference in the respective oxygen levels determined for each of the first and second locations. In an embodiment, the comparison is to determine whether a greater oxygen level was determined for the first location or the second location. In an embodiment, the comparison is to determine an extent of difference between the oxygen levels determined for the first and second locations. In an embodiment, one or both of the oxygen levels determined for the first location or the second location can be adjusted based on a location at which the respective oxygen level was detected. Adjustment of the oxygen level can be based on location-based differences oxygen level. Non-limiting examples of location-based differences in oxygen levels include an order or amount of blood flow to the location where the measurement is taken, or the like. In an embodiment, adjustment of the oxygen level based on the measurement location can be based on historical data for the subject. In an embodiment, adjustment of the oxygen level can be based on a database including historical data from a plurality of subjects. In an embodiment, adjusting the oxygen level based on the measurement location can be based on a model for determining expected differences in detected analyte amounts by location, for example models of blood flow and/or oxygen consumption at particular locations in the body. For example, the adjustment of the oxygen levels can reduce or remove location-related differences such that results of the detection of the oxygen levels can be presented on a similar scale or as comparable values. In embodiments, results of detection of the oxygen levels at additional different locations from the first or second locations can further be included in the comparison performed at 94.

A condition of the subject can be determined based on the comparison at 96. The condition can be based on the particular one or more analytes and the relationship thereof determined by the comparison at 94. The condition can further be based on the particular first and second locations where the oxygen levels are detected. The condition can be any suitable condition that relationships among oxygen levels at first and second locations are indicative of, such as circulatory issues, tumor growth, organ function, or the like. In an embodiment, the condition can be determined based on similarity between the detected oxygen levels or trends over time for those oxygen levels. For example, consistent oxygen levels or consistent trends or patterns in variations in oxygen levels at each of two or more body locations can be indicative of system-wide conditions or characteristics of the subject. For example, where the oxygen levels vary periodically with similar or the same periodicity at both the first and second locations, conditions producing body-wide systematic variations, such as heart rates, respiration rates, and the like can be determined based on the oxygen levels.

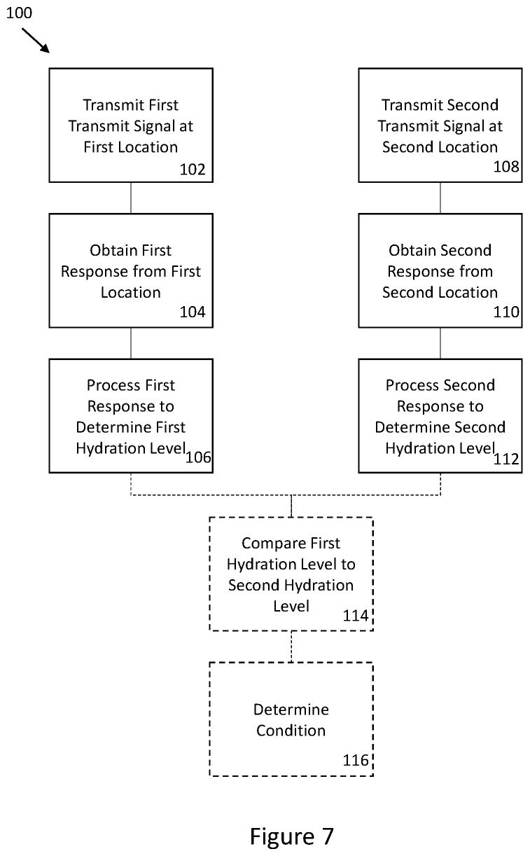

FIG. 7 shows a flowchart of a method for determining a hydration level at a plurality of locations in a subject. Method 100 includes transmitting a first transmit signal into the subject at a first location at 102, obtaining a first response signal from the first location at 104, and processing the first response signal to determine a hydration level at the first location at 106. Method 100 further includes transmitting a second transmit signal into the subject at a second location at 108, obtaining a second response signal from the second location at 110, and processing the second response signal to determine a hydration level at the second location at 112. Method 110 can further include comparing the hydration level at the first location and the hydration level at the second location at 114 and determining a condition of the subject based on the comparison at 116.

A hydration level is determined at a first location by transmitting a first transmit signal into the subject at a first location at 102, obtaining a first response signal from the first location at 104, and processing the first response signal to determine the hydration level at the first location at 106. The first location can be any location on a body of the subject, for example a section of the body. Non-limiting examples of sections of the body that can be selected as the first location include the lower left leg, upper left leg, lower right leg, upper right leg, lower left arm, upper left arm, groin, abdomen, chest, neck, or the head. The first location can be selected based on convenience, comfort, or availability of the location on the subject. The first location can be selected to correspond to an organ or tissue of interest, such as the kidneys, the bladder, the mouth, the brain, the lungs, specific muscles, fluid accumulations in the body, blood flowing through specific regions of the body or specific blood vessels, or the like. The first location can be selected to provide a baseline measurement for comparison to the detection of the hydration level at the second location. The first transmit signal can be generated by a transmit circuit of a sensor, such as transmit circuit 15 of sensor 5 as described above and shown in FIG. 1 and then transmitted into the subject at the first location 102. The first transmit signal can be transmitted into the first location by a transmit antenna of the sensor, such as transmit antenna 11 as described above and shown in FIG. 1. A first response signal is obtained from the first location on the subject at 104. The first response signal results from the transmitting of the first transmit signal into the subject at the first location 104. The first response signal can be obtained 104 using a receive antenna of the sensor such as receive antenna 13 of sensor 5 as described above and shown in FIG. 1.

The first response signal obtained at 104 is processed to determine a first hydration level at the first location at 106. In an embodiment, the first response signal obtained at 104 is processed at 106 to determine an amount of water detected at the first location. In an embodiment, the first response signal obtained at 104 is processed at 106 to determine amounts of one or more analytes used to determine the hydration level at the first location. The one or more analytes can be, for example, hemoglobin, red blood cells as a whole, one or more hormones, sodium, one or more solutes from which osmolarity can be determined, or the like. The amount of the analytes can be used to determine one or more indicia of hydration, such as concentrations of one or more analytes, hematocrit, osmolarity, or any other suitable measurement of a hydration level of the subject. The osmolarity can be an osmolarity of one or more of plasma, interstitial fluid, saliva, urine, or the like.

A hydration level is determined at a second location by transmitting a second transmit signal into the subject at a second location at 108, obtaining a second response signal from the second location at 110, and processing the second response signal to determine a second hydration level at the second location at 112. The second location can be any distinct location on the subject different from the first location. The second location can be selected based on convenience, comfort, or availability of the location on the subject. The second location can be selected to correspond to an organ or tissue of interest, such as the kidneys, the bladder, the mouth, the brain, the lungs, specific muscles, fluid accumulations in the body, blood flowing through specific regions of the body or specific blood vessels, or the like. The second location can be selected to provide a baseline measurement for comparison to the detection of the hydration level at the first location. The second transmit signal can be generated by a transmit circuit of a sensor, such as transmit circuit 15 of sensor 5 as described above and shown in FIG. 1 and then transmitted into the subject at the second location 108. The second transmit signal can be transmitted into the second location by a transmit antenna of the sensor, such as transmit antenna 11 as described above and shown in FIG. 1. A second response signal is obtained from the second location on the subject at 110. The second response signal results from the transmitting of the second transmit signal into the subject at the second location 108. The second response signal can be obtained 110 using a receive antenna of the sensor such as receive antenna 13 of sensor 5 as described above and shown in FIG. 1.

The second response signal obtained at 110 is processed to determine a second hydration level at the second location at 112. In an embodiment, the second response signal obtained at 110 is processed at 112 to determine an amount of water detected at the first location. In an embodiment, the first response signal obtained at 110 is processed at 112 to determine amounts of one or more analytes used to determine the hydration level at the first location. The one or more analytes can be, for example, hemoglobin, red blood cells as a whole, one or more hormones, sodium, one or more solutes from which osmolarity can be determined, or the like. The amount of the analytes can be used to determine one or more indicia of hydration, such as concentrations of one or more analytes, hematocrit, osmolarity, or any other suitable measurement of a hydration level of the subject. The osmolarity can be an osmolarity of one or more of plasma, interstitial fluid, saliva, urine, or the like.

In an embodiment, determination of the hydration level at the first location and the determination of the hydration level at the second location can be performed at overlapping times. In an embodiment, determination of the hydration level at the first location and the determination of the hydration level at the second location can be performed at separate times. In an embodiment, determination of the hydration level at the first location and the determination of the hydration level at the second location can be performed sequentially.

The hydration levels at the first and second locations on the subject can be compared at 114. In an embodiment, the comparison is to determine if there is a difference in the respective hydration levels determined for each of the first and second locations. In an embodiment, the comparison is to determine whether a greater hydration level was determined for the first location or the second location. In an embodiment, the comparison is to determine an extent of difference between the hydration levels determined for the first and second locations. In an embodiment, one or both of the hydration levels determined for the first location or the second location can be adjusted based on a location at which the respective hydration level was detected. Adjustment of the hydration levels can be based on location-based differences in hydration level. Non-limiting examples of location-based differences in hydration levels include an order or amount of blood flow to the location where the measurement is taken, or the like. In an embodiment, adjustment of the hydration level based on the measurement location can be based on historical data for the subject. In an embodiment, adjustment of the hydration level can be based on a database including historical data from a plurality of subjects. In an embodiment, adjusting the hydration level based on the measurement location can be based on a model for determining expected differences in detected analyte amounts by location, for example models of blood flow, fluid accumulation, or the like at particular locations in the body. For example, the adjustment of the hydration levels can reduce or remove location-related differences such that results of the detection of the hydration levels can be presented on a similar scale or as comparable values. In embodiments, results of detection of the hydration levels at additional different locations from the first or second locations can further be included in the comparison performed at 114.

A condition of the subject can be determined based on the comparison of hydration levels at 116. The condition can be based on the relationships between the hydration levels determined by the comparison at 114. The condition can further be based on the particular first and second locations where the hydration levels are detected. The condition can be any suitable condition that relationships among hydration levels at first and second locations are indicative of, such as circulatory issues, kidney issues, accumulation of fluids such as edema, local deficiencies of fluids such as conditions giving rise to bedsores, and the like. In an embodiment, the condition can be determined based on similarity between the detected hydration levels or trends over time for those hydration levels. For example, consistent hydration levels or consistent trends or patterns in variations in hydration levels at each of two or more body locations can be indicative of system-wide conditions or characteristics of the subject. For example, where the hydration levels vary periodically with similar or the same periodicity at both the first and second locations, conditions producing body-wide systematic variations, such as water intake, cycles of kidney function, loss of water due to sweating, and the like can be determined.

In an embodiment, hydration levels determined in the subject through method 100 can be used to adjust or modify other determinations of amounts of analytes or biological parameters. For example, hydration levels can be used to normalize determinations of concentrations of analytes, to adjust determined oxygen levels, or the like. In an embodiment, method 100 and method 80 can be combined, with the determined hydration levels from 106 and 112 in turn used as factors in the determination of oxygen levels at 86 and 92 of method 80. In an embodiment, hydration levels determined in the subject can be included in determining a state of the subject, for example as part of a set of analytes and/or biological parameters used as indicators of kidney function or disease.

The examples disclosed in this application are to be considered in all respects as illustrative and not limitative. The scope of the invention is indicated by the appended claims rather than by the foregoing description; and all changes which come within the meaning and range of equivalency of the claims are intended to be embraced therein.

1. A method of detecting levels of a biological parameter in a subject, comprising:
detecting a first level of the biological parameter at a first location on a body of the subject; and
detecting a second level of the biological parameter at a second location on the body of the subject, the second location different from the first location;
wherein detecting the first level of the biological parameter includes:
generating a first transmit signal in a radio or microwave frequency range of the electromagnetic spectrum;
transmitting the first transmit signal into the subject at the first location;
detecting a first response resulting from transmitting the first transmit signal into the subject at the first location; and
processing the first response to determine the first level of the biological parameter, and
detecting the second level of the biological parameter includes:
generating a second transmit signal in a radio or microwave frequency range of the electromagnetic spectrum;
transmitting the second transmit signal into the subject at the second location;
detecting a second response resulting from transmitting the second transmit signal into the subject at the second location; and
processing the second response to determine the second level of the biological parameter,
wherein the biological parameter includes at least one of an oxygen level or a hydration level.

2. The method of claim 1, further comprising determining a condition of the subject based on a differential between the first level of the biological parameter and the second level of the biological parameter.

3. The method of claim 1, wherein detecting the first level of the biological parameter is performed using a first sensor and detecting the second level of the biological parameter is performed using a second sensor.

4. The method of claim 3, wherein detecting the first level of the biological parameter and detecting the second level of the biological parameter overlap in time.

5. The method of claim 1, wherein detecting the first level of the biological parameter includes positioning a sensor at the first location and wherein detecting the second level of the biological parameter includes positioning said sensor at the second location.

6. The method of claim 1, wherein the biological parameter includes the oxygen level.

7. The method of claim 6, wherein the oxygen level is determined based on the hydration level.

8. The method of claim 1, wherein the biological parameter includes the hydration level.

9. The method of claim 1, further comprising detecting a third level of the biological parameter at a third location on the body of the subject, different from the first location and the second location.

10. A system for detecting levels of a biological parameter in a subject, comprising:
a first sensor, comprising:
an antenna array having at least one transmit antenna and at least one receive antenna, the at least one transmit antenna is positioned and arranged to transmit a transmit signal into a target, and the at least one receive antenna is positioned and arranged to detect a response resulting from transmission of the transmit signal by the at least one transmit antenna into the target;
a transmit circuit that is electrically connectable to the at least one transmit antenna, the transmit circuit is configured to generate a transmit signal to be transmitted by the at least one transmit antenna, the transmit signal is in a radio or microwave frequency range of the electromagnetic spectrum; and
a receive circuit that is electrically connectable to the at least one receive antenna, the receive circuit is configured to receive a response detected by the at least one receive antenna,
a controller configured to:
process the response detected by the at least one receive antenna at a first location to determine a first level of the biological parameter,
receive results of detection of a second level of the biological parameter taken at a second location on the body of the subject, the second location different from the first location, and
compare the first level to the second level,
wherein the biological parameter includes at least one of an oxygen level and a hydration level.

11. The system of claim 10, further comprising a second sensor, the second sensor comprising:
a second sensor antenna array having at least one second sensor transmit antenna and at least one second sensor receive antenna, the at least one second sensor transmit antenna is positioned and arranged to transmit a second sensor transmit signal into a second target, and the at least one second sensor receive antenna is positioned and arranged to detect a response resulting from transmission of the second sensor transmit signal by the at least one second sensor transmit antenna into the second target, the second target being at the second location on the body of the subject;
a second sensor transmit circuit that is electrically connectable to the at least one second sensor transmit antenna, the second sensor transmit circuit is configured to generate a second sensor transmit signal to be transmitted by the at least one second sensor transmit antenna, the second sensor transmit signal is in a radio or microwave frequency range of the electromagnetic spectrum; and
a second sensor receive circuit that is electrically connectable to the at least one second sensor receive antenna, the second sensor receive circuit configured to receive the response detected by the at least one second sensor receive antenna,
wherein the controller is configured to receive the results of detection of the second level of the biological parameter from the second sensor.

12. The system of claim 10, wherein the controller is configured to receive the results of detection of the second level of the biological parameter from the first sensor.

13. The system of claim 10, wherein the controller is configured to determine a condition based on a differential between the first level of the biological parameter and the second level of the biological parameter.

14. The system of claim 10, wherein the controller is included in a device including the first sensor.

15. The system of claim 10, wherein the controller is included in a remote device, separate from the first sensor.

16. The system of claim 10, wherein the controller is included in a cloud server.

17. The system of claim 10, wherein the results of detection of the second level of the biological parameter received by the controller include a second amount of each of the one or more analytes detected at the second location, and the controller is further configured to process the second amount to determine the second oxygen level.

18. The system of claim 10, wherein the biological parameter includes the oxygen level.

19. The system of claim 18, wherein the controller is configured to determine the oxygen level based on the hydration level.

20. The system of claim 10, wherein the biological parameter includes the hydration level.
